(19) United States
(12) Patent Application Publication
(10) Pub. No.: US 2012/0178827 A1
(43) Pub. Date: Jul. 12, 2012

(54) Solid Pharmaceutical Preparations Comprising Amphiphilic Copolymers On The Basis Of Polyethers In Combination With Surfactants

(75) Inventors: Karl Kolter, Limburgerhof (DE); Dejan Djuric, Mannheim (DE); Stefan Fischer, Freinsheim (DE)

(73) Assignee: BASF SE, Ludwigshafen (DE)

(21) Appl. No.: 13/496,566

(22) PCT Filed: Sep. 13, 2010
(86) PCT No.: PCT/EP10/63369

§ 371 Date: Mar. 16, 2012

(30) Foreign Application Priority Data
Sep. 18, 2009 (EP) 09170706.7

(51) Int. Cl. A61K 47/34

(52) U.S. Cl. 514/772.7

(57) ABSTRACT

Dosage forms comprising formulations of sparingly water-soluble active ingredients in a polymer matrix composed of polyether copolymers, said polyether copolymers being obtained by free-radically initiated polymerization of a mixture of 30 to 80% by weight of N-vinyllactam, 10 to 50% by weight of vinyl acetate and 10 to 50% by weight of a polyether, and of at least one surfactant, in which the sparingly water-soluble active ingredient is present in amorphous form in the polymer matrix.

The present invention relates to solid pharmaceutical formulations of amphiphilic copolymers and sparingly water-soluble active ingredients in combination with surfactants which are capable of influencing the stability of the formulation and/or the release of the active ingredients.

The amphiphilic copolymers used are especially copolymers which are obtainable by polymerizing N-vinyllactam and vinyl acetate in the presence of polyethers.

The corresponding copolymers based on polyethers function as solubilizers for the sparingly water-soluble biologically active substances.

In the production of homogeneous formulations, especially of biologically active substances, the solubilization of hydrophobic, i.e. sparingly water-soluble, substances has gained very great practical significance.

Solubilization is understood to mean the solubilizing of substances which are sparingly soluble or insoluble in a particular solvent, especially water, by interface-active compounds, the solubilizers. Such solubilizers are capable of converting sparingly water-soluble or water-insoluble substances to clear, at most opalescent, aqueous solutions, without the chemical structure of these substances undergoing any change in the process.

In the solubilizates produced, the sparingly water-soluble or water-insoluble substance is present in colloidally dissolved form in the molecular associates of the surface-active compounds which form in aqueous solution, for example, hydrophobic domains or micelles. The resulting solutions are stable or metastable monophasic systems with a visually clear to opalescent appearance.

In the case of pharmaceutical formulations, the bioavailability and hence the action of medicaments can be enhanced by the use of solubilizers.

A further desirable requirement on solubilizers is the ability to form so-called “solid solutions” with sparingly soluble substances. The term “solid solution” describes a state in which a substance is distributed in colloidal dispersion or ideally molecular dispersion in a solid matrix, for example, a polymer matrix. Such solid solutions lead, for example, when used in solid pharmaceutical administration forms of a sparingly soluble active ingredient, to improved release of the active ingredient. An important requirement on such solid solutions is that they are stable over a long period even in the course of storage, which means that the active ingredient does not crystallize out. Moreover, the capacity of the solid solution or, in other words, the ability to form stable solid solutions with maximum active ingredient contents is also of significance.

WO 2007/051743 discloses the use of water-soluble or water-dispersible copolymers of N-vinyllactam, vinyl acetate and polyethers as solubilizers for pharmaceutical, cosmetic, food technology, agrochemical or other industrial applications. It is described in quite general terms therein that the corresponding graft polymers can also be processed with the active ingredients in the melt.

WO 2009/013202 likewise discloses that graft polymers of N-vinyllactam, vinyl acetate and polyethers can be used to solubilize sparingly soluble active ingredients, by melting the graft polymers in an extruder and mixing them with pulverulent or liquid active ingredients, the extrusion being described at temperatures significantly below the melting point of the active ingredient.

However, the problem of thermal stress on the active ingredient should always be considered in extrusion processes. In this respect, there was still room for improvement in the procedure.

It was an object of the present invention to find an improved process and hence improved formulations of sparingly water-soluble active ingredients which, coupled with good bioavailability, enable stable release.

Accordingly, a process for producing formulations of sparingly water-soluble biologically active substances in a polymer matrix based on amphiphilic copolymers which are obtained by free-radically initiated polymerization of a mixture of
i) 30 to 80% by weight of N-vinyllactam,
ii) 10 to 50% by weight of vinyl acetate, and
iii) 10 to 50% by weight of a polyether,
   with the proviso that the sum of i), ii) and iii) equals 100% by weight, has been found, wherein surfactants are added during processing.

In one embodiment of the invention, preferred polymers obtained from:
i) 30 to 70% by weight of N-vinyllactam,
ii) 15 to 35% by weight of vinyl acetate, and
iii) 10 to 35% by weight of a polyether, are used.

Polymers used with particular preference are obtainable from:
i) 40 to 60% by weight of N-vinyllactam,
ii) 15 to 35% by weight of vinyl acetate, and
iii) 10 to 30% by weight of a polyether.

Polymers used with very particular preference are obtainable from
i) 50 to 60% by weight of N-vinyllactam,
ii) 25 to 35% by weight of vinyl acetate, and
iii) 10 to 20% by weight of a polyether.

For the preferred and particularly preferred compositions too, the proviso applies that the sum of components i), ii), and iii) equals 100% by weight.

Useful N-vinyllactam includes N-vinylcaprolactam or N-vinylpyrrolidone or mixtures thereof. Preference is given to using N-vinylcaprolactam.

The graft bases used are polyethers. Useful polyethers are preferably polyalkylene glycols. The polyalkylene glycols may have molecular weights of 1000 to 100 000 D [daltons], preferably 1500 to 35 000 D, more preferably 1500 to 10 000 D. The molecular weights are determined proceeding from the OH number measured to DIN 53240.

Particularly preferred polyalkylene glycols include polyethylene glycols. Also additionally suitable are polypropylene glycols, polytetrahydrofurans or polybutylene glycols, which are obtained from 2-ethyloxirane or 2,3-dimethyloxirane.

Suitable polyethers are also random or block copolymers of polyalkylene glycols obtained from ethylene oxide, propylene oxide and butylene oxides, for example polyethylene glycol-polypropylene glycol block copolymers. The block copolymers may be of the AB type or of the ABA type.

The preferred polyalkylene glycols also include those which are alkylated at one or both OH end groups. Useful alkyl radicals include branched or unbranched C₁- to C₂₂-alkyl radicals, preferably C₁-C₁₈-alkyl radicals, for example, methyl, ethyl, n-butyl, isobutyl, pentyl, hexyl, octyl, nonyl, decyl, dodecyl, tridecyl or octadecyl radicals.

General processes for preparing the inventive graft copolymers are known per se. They are prepared by free-radically initiated polymerization, preferably in solution, in nonaqueous organic solvents or in mixed nonaqueous/aqueous solvents. Suitable preparation processes are described, for example, in WO 2007/051743 and WO 2009/013202, the disclosure of which is referred to explicitly with regard to the preparation process.

In principle, suitable surfactants are all of those which have an HLB value greater than 3, preferably greater than 6, more preferably greater than 10 (see Fiedler, Lexikon der Hilfsstoffe [Lexicon of Excipients]). Suitable in principle are anionic, cationic, nonionic, zwitterionic or amphiphilic surfactants.

Particularly suitable surfactants are:
alpha-tocopherol polyethylene glycol succinate, stearic acid and salts thereof, glyceryl monostearate, ethoxylated glyceryl monostearate, sorbitan laurate, sorbitan monooleate, Ceteareth-20 (cetylstearyl alcohol×20 mol of ethylene oxide units), sodium laurylsulfate, docusate sodium, poloxamers, ethoxylated castor oil, hydrogenated ethoxylated castor oil, Macrogol fatty alcohol ethers, Macrogol fatty acid esters, Macrogol sorbitan fatty alcohol ethers and Macrogol sorbitan fatty acid esters, Polysorbate 20, 40, 60, 80, Span® and Tween® products.

Suitable substances are ionic and nonionic surfactants, for example Solutol® HS 15 (Macrogol-15 hydroxystearate), Tween® 80, polyoxyethylated fatty acid derivatives such as Cremophor®RH40 (polyoxyl 40 Hydrogenated Castor Oil, USP), Cremophor EL (Polyoxyl 35 Castor Oil, USP), poloxamers, docusate sodium or sodium laurylsulfate.

The surfactants are used in the extrudate in a concentration between 0.1 and 80% by weight, preferably 0.2 and 50% by weight and more preferably 0.5 and 30% by weight.

The solid formulations can be produced by methods known per se. In a preferred procedure, the solid formulations are produced by extrusion.

The polymers can be supplied to the extruder either in pulverulent form or in the form of solutions or dispersions.

The dispersions or solutions of the polymer can be converted to solid form by removing the dispersant or solvent in the extruder in the molten state and cooling the melt.

The melt thus obtained can then be cooled and granulated. This is done by so-called hot-cutting or cooling under air or protective gas, for example, on a Teflon or chain belt and subsequent granulation of the cooled melt extrudate. However, cooling is also possible in a solvent in which the polymers do not have significant solubility.

The following methods A-E can be used in principle:
physical powder mixing of polymers and active ingredient, and supply of this powder mixture in the extruder; supply of the active ingredient via a separate bypass into the unmolten polymer mixture; supply of the active ingredient via a side feed into molten polymers; polymer dissolution with active ingredient dispersed or dissolved therein in partly degassed polymer melt or unmelted polymer mixture; additionally introducing solvent into the extruder and evaporating it again.

For the process according to the invention, suitable extruder types in principle are the customary extruder types known to those skilled in the art. Typically, these comprise a housing, a drive unit with transmission, and a process unit which consists of the extruder shaft or shafts equipped with the screw elements, modular construction being assumed in this case.

The extruder consists of a plurality of sections, which are each assigned to particular process units. Each of these sections consists of one or more barrels (barrel blocks) as the smallest independent unit and the corresponding screw sections with the screw elements corresponding to the process task.

The individual barrels should be heatable. In addition, the barrels may also be designed for cooling, for example, for cooling with water. The individual barrel blocks are preferably independently heatable and coolable, such that different temperature zones can also be established along the extrusion direction.

The extruder is advantageously configured as a corotatory twin screw extruder. The screw configuration may have different shear levels according to the product. The screw configuration can be matched to the particular requirements, according to the composition of the formulation, with the customary variable construction elements such as conveying elements, kneading elements, backup elements and the like.

Suitable twin screw extruders may have a screw diameter of 16 to 70 mm and a length of 25 to 40 D.

The entire extruder is formed from barrel blocks, whose temperatures can be controlled individually. The first two barrels may be temperature-controlled for the purpose of better material intake. From the third barrel, a constant temperature is preferably established, which should be selected specifically to the material and depends especially on the melting point of the active ingredient used and the glass transition temperature of the polymer. The resulting product temperature typically, however, depends on the shear level of the screw element used and may in some cases be 20-30° C. higher than the barrel temperature established.

The melting zone may be followed downstream by a venting zone, which is advantageously operated at ambient pressure.

The round dies used may have a diameter of 0.5 to 5 mm. Other die forms such as slot dies may likewise be used, in particular when a greater material throughput is desired.

The resulting extrudates can be processed with a granulator to pellets which can in turn be comminuted (ground) further to a powder. The pellets or powder can be filled into capsules or pressed to tablets using customary tableting assistants. In this context, it is also possible to use further release-controlling assistants.

In addition, it is possible to use, during the extrusion, water, organic solvents, buffer substances or plasticizers. Especially water or volatile alcohols are options for this purpose. This process enables reaction at relatively low temperature. The amounts of solvent or plasticizer are typically between 0 and 30% of the extrudable material. The water or solvent can already be removed by a venting point in the extruder at standard pressure, or by applying reduced pressure. Alternatively, these components evaporate when the extrudate leaves the extruder and the pressure is reduced to standard pressure. In the case of less volatile components, the extrudate can correspondingly be dried subsequently.

In a particular variant of the production process, directly after the extrusion, the thermoplastic material is calendered to a tablet-like compact which constitutes the ultimate administration form. In this variant, it may be advisable to add further constituents, for example polymers for adjusting the glass transition temperature and the melt viscosity, disintegrants, further solubilizers, plasticizers, dyes, flavorings, sweeteners, etc. actually before or during the extrusion. In principle, these substances can also be used when the extrudate is first comminuted and then pressed to tablets.

To adjust the glass transition temperature of the formulation, additionally water-soluble polymers with a high glass transition temperature, for example polyvinylpyrrolidone with K values of 17-120, hydroxyalkyl celluloses or hydroxyalkyl starches can be used. Too high a glass transition temperature of the formulation can be lowered by adding plasticizers. Suitable plasticizers for this purpose are in principle all plasticizers which are also used for pharmaceutical coatings, for example triethyl citrate, tributyl citrate, acetyltributyl citrate, triacetin, propylene glycol, polyethylene glycol 400, dibutyl sebacate, glyceryl monostearate, lauric acid, cetylstearyl alcohol.

The still plastic mixture is preferably extruded through a die, cooled and comminuted. Suitable comminution methods are in principle all known techniques customary therefor, such as hot or cold cutting.

The extrudate is cut, for example, with rotating blades or with an air jet and then cooled with air or under protective gas.

It is also possible to lay the extrudate as a melt strand on a cooled belt (stainless steel, Teflon, chain belt) and to granulate it after solidification.

Subsequently, the extrudate can optionally be ground. The formulations are obtained as free-flowing water-soluble powders. Preference is given to establishing particle sizes of 20 to 250 μm.

In addition, it is also possible to process the plastic mixture of polymer and active substance by injection molding.

The formulations obtained by the process according to the invention can in principle be used in all fields in which only sparingly water-soluble or water-insoluble substances are either to be used in aqueous formulations or are to display their action in an aqueous medium.

According to the invention, the term “sparingly water-soluble” also comprises virtually insoluble substances and means that, for a solution of the substance in water at 20° C. at least 30 to 100 g of water is required per g of substance. In the case of virtually insoluble substances, at least 10 000 g of water are required per g of substance.

In the context of the present invention, sparingly-water soluble substances are preferably understood to mean biologically active substances such as active pharmaceutical ingredients for humans and animals, active cosmetic or agrochemical ingredients, or food supplements or active dietetic ingredients.

In addition, useful sparingly soluble substances to be solubilized also include dyes such as inorganic or organic pigments.

According to the invention, useful biologically active substances include, in principle, all solid active ingredients which have a melting point below the decomposition point under extrusion conditions of the copolymers. The copolymers can generally be extruded at temperatures up to 260° C. The lower temperature limit is guided by the composition of the mixtures to be extruded and the sparingly soluble substances to be processed in each case.

The active pharmaceutical ingredients used are water-insoluble substances or substances with low water solubility according to the DAB 9 definition already mentioned.

The active ingredients may come from any indication sector.

Examples here include benzodiazepines, antihypertensives, vitamins, cytostatics—especially taxol, anesthetics, neuroleptics, antidepressives, antivirals, for example anti-HIV drugs, antibiotics, antimycotics, antidementives, fungicides, chemotherapeutics, urologics, thrombocyte aggregation inhibitors, sulfonamides, spasmolytics, hormones, immunoglobulins, sera, thyroid therapeutics, psychopharmaceuticals, Parkinson's drugs and other antihyperkinetics, ophthalmics, neuropathy preparations, calcium metabolism regulators, muscle relaxants, anesthetics, lipid-lowering drugs, liver therapeutics, coronary drugs, cardiac drugs, immunotherapeutics, regulatory peptides and inhibitors thereof, hypnotics, sedatives, gynaecologicals, gout remedies, fibrinolytics, enzyme preparations and transport proteins, enzyme inhibitors, emetics, blood-flow stimulators, diuretics, diagnostics, corticoids, cholinergics, biliary therapeutics, antiasthmatics, bronchodilators, beta-receptor blockers, calcium antagonists, ACE inhibitors, arteriosclerotic drugs, anti-inflammation drugs, anticoagulants, antihypotensives, antihypoglycemics, antihypertensives, antifibrinolytics, antiepileptics, antiemetics, antidotes, antidiabetics, antiarrhythmics, antianemics, antiallergics, anthelmintics, analgesics, analeptics, aldosterone antagonists, slimming drugs.

Among the abovementioned pharmaceutical formulations, particular preference is given to those which are orally administrable formulations.

The content of inventive solubilizer in the pharmaceutical formulation is, depending on the active ingredient, in the range from 1 to 75% by weight, preferably 5 to 60% by weight, more preferably 5 to 50% by weight.

To produce pharmaceutical administration forms, for example, tablets, the extrudates can be admixed with customary pharmaceutical excipients.

These are substances form the class of the fillers, plasticizers, solubilizers, binders, silicates and disintegrants and adsorbents, lubricants, flow agents, dyes, stabilizers such as antioxidants, wetting agents, preservatives, mold release agents, aromas or sweeteners, preferably fillers, plasticizers and solubilizers.

The fillers added may, for example, be inorganic fillers such as oxides of magnesium, aluminum, silicon, titanium carbonate or calcium carbonate, calcium phosphate or magnesium phosphate or organic fillers such as lactose, sucrose, sorbitol, mannitol.

Suitable plasticizers are, for example, triacetin, triethyl citrate, glyceryl monostearate, low molecular weight polyethylene glycols or poloxamers.

Suitable additional solubilizers are interface-active substances with an HLB (Hydrophilic Lipophilic Balance) value greater than 11, for example hydrogenated castor oil ethoxylated with 40 ethylene oxide units (Cremophor® RH 40), castor oil ethoxylated with 35 ethylene oxide units (Cremophor EL), Polysorbate 80, poloxamers or sodium laurylsulfate.

The lubricants used may be stearates of aluminum, calcium, magnesium and tin, and also magnesium silicate, silicones and the like.

The flow agents used may, for example, be talc or colloidal silicon dioxide.

A suitable binder is, for example, microcrystalline cellulose.

The disintegrants may be crosslinked polyvinylpyrrolidone or crosslinked sodium carboxymethyl starch. Stabilizers may be ascorbic acid or tocopherol.

Dyes are, for example, iron oxides, titanium dioxide, triphenylmethane dyes, azo dyes, quinoline dyes, indigotin dyes, carotenoids, in order to dye the administration forms, opacifiers, such as titanium dioxide or talc, in order to increase the transparency and to save dyes.

In addition to use in cosmetics and pharmacy, the formulations produced in accordance with the invention are also suitable for use in the foods sector, for example, for the incorporation of sparingly water-soluble or water-insoluble nutrients, assistants or additives, for example, fat-soluble vitamins or carotenoids. Examples include drinks, colored with carotenoids.

The use of the formulations obtained in accordance with the invention in agrochemistry may include formulations which comprise pesticides, herbicides, fungicides or insecticides, and in particular also those formulations of crop protection compositions which are used as formulations for spraying or watering.

With the aid of the process according to the invention, it is possible to obtain so-called solid solutions comprising sparingly soluble substances. Solid solutions refer in accordance with the invention to systems in which no crystalline components of the sparingly soluble substance are observed.

The addition of surfactants with a wide variety of different HLB values enables the lowering of the viscosity of the extrusion melt. This allows the extrusion temperature to be lowered and the resulting extrudate to be subjected to less thermal stress overall during the process. By virtue of the lowering of the extrusion temperature, entirely colorless extrudates are also obtainable, and it is possible to process active ingredients which are very thermally sensitive. The extrusion is preferably effected below the melting point of the active ingredients.

In addition, the wetting can be improved by the use of surfactants, such that the active ingredient is released more rapidly. This is important in particular when high proportions of hydrophobic active ingredient which lipophilize the entire matrix and hence make the formulation more difficult to wet are incorporated.

On visual assessment of the stable solid solutions, no amorphous constituents are evident. The visual assessment can be effected with a light microscope either with or without a polarization filter at 40-fold magnification.

In addition, the formulations can also be examined for crystallinity or amorphicity with the aid of XRD (X-Ray Diffraction) and DSC (Differential Scanning Calorimetry).

The formulations obtained by the process according to the invention are, as stated, present in amorphous form which means that the crystalline components of the biologically active substance are less than 5% by weight. The amorphous state is preferably checked by means of DSC or XRD. Such an amorphous state can also be referred to as an X-ray-amorphous state.

The process according to the invention allows the production of stable formulations with a high active ingredient loading and good stability with regard to the amorphous state of the sparingly soluble substance.

The process according to the invention allows the production of stable formulations with high active ingredient loading.

### EXAMPLES

### Preparation of the Copolymer

In a stirred apparatus, the initial charge without the portion from feed 2 was heated to 77° C. under an N₂ atmosphere. When the internal temperature of 77° C. had been attained the portion from feed 2 was added and partly polymerized for 15 min. Subsequently, feed 1 was metered in within 5 h and feed 2 within 2 h. Once all feeds had been metered in, the reaction mixture was polymerized for a further 3 h. After the further polymerization, the solution was adjusted to a solids content of 50% by weight.

Initial charge: 25 g of ethyl acetate
104.0 g of PEG 6000
1.0 g of feed 2

Feed 1: 240 g of vinyl acetate
456 g of vinylcaprolactam
240 g of ethyl acetate

Feed 2: 10.44 g of tert-butyl perpivalate (75% by weight in aliphatics mixture) 67.90 g of ethyl acetate

Subsequently, the solvent was removed by a spray process to obtain a pulverulent product. The K value was 16, measured in 1% by weight solution in ethanol.

The twin screw extruder which was used for the production of the formulations described in the examples which follow had a screw diameter of 16 mm and a length of 40 D. The entire extruder was formed from 8 individually temperature-controllable barrel blocks.

The solid solutions produced were examined by means of XRD (X-Ray Diffractometry) and DSC (Differential Scanning Calorimetry) for crystallinity and amorphicity using the following equipment and conditions:

### XRD

Instrument: D 8 Advance diffractometer with 9-tube sample changer (from Bruker/AXS)
Measurement method: θ-θ geometry in reflection
2 theta angle range: 2-80°
Step width: 0.02°
Measurement time per angle step: 4.8 s
Divergence slit: Göbel mirror with 0.4 mm inserted aperture
Antiscattering slit: Soller slit
Detector: Sol-X detector
Temperature: Room temperature
Generator setting: 40 kV/50 mA

### DSC

DSC Q 2000 from TA Instruments
Parameters:
Starting weight approx. 8.5 mg
Heating rate: 20K/min

The active ingredient release was effected using USP apparatus (paddle method) 2, 37° C., 50 rpm (BTWS 600, Pharmatest). The extrudate strands were divided by means of a pelletizer to a length of 3 mm and introduced into hard gelatin capsules. The active ingredient released was detected by UV spectroscopy (Lambda-2, Perkin Elmer).

### Example 1

1600 g of copolymer, 50 g of SDS and 400 g of itraconazole (melting point 166° C.) were weighed into a Turbula mixing vessel and mixed in a T10B Turbula mixer for 10 minutes.

The mixture was extruded under the following conditions:
Zone temperature of 1st barrel: 20° C.; 2nd barrel: 40° C.
Zone temperature from the 3rd barrel: 140° C.
Screw speed 200 rpm
Throughput: 600 g/h
Die diameter 3 mm

The addition of SDS lowered the viscosity of the extrudate, such that the extrusion temperature could be lowered by 20° C. to 130° C. After 45 minutes in 0.1 normal HCl, 100% active ingredient had been released.

### Example 2

1600 g of copolymer and 400 g of felodipin (melting point 145° C.) were weighed into a Turbula mixing vessel and mixed in a T10B Turbula mixer for 10 minutes. A reciprocating piston pump was used to feed docusate sodium (10% solution) into the extruder.

The mixture was extruded under the following conditions:
Zone temperature of 1st barrel: 20° C.; 2nd barrel: 40° C.
Zone temperature from the 3rd barrel: 120° C.
Screw speed 100 rpm
Throughput: 800 g/h
Liquid feed rate: 100 g/h
Die diameter 3 mm

The solid solutions were examined by XRD and by DSC and were found to be amorphous. The release of the active ingredient in pH 6.8 phosphate buffer was 31% after 2 h; after 10 h, 79% of the active ingredient originally used had been released.

### Example 3

1600 g of copolymer and 400 g of danazol (melting point 225° C.) were weighed into a Turbula mixing vessel and mixed in a T10B Turbula mixer for 10 minutes. A reciprocating piston pump was used to feed Solutol HS 15 into the extruder.

The mixture was extruded under the following conditions:
Zone temperature of 1st barrel: 20° C.; 2nd barrel: 40° C.
Zone temperature from the 3rd barrel: 400° C.
Screw speed 100 rpm
Throughput: 800 g/h
Liquid feed rate: 50 g/h
Die diameter 3 mm

The solid solutions were examined by XRD and by DSC and were found to be amorphous. The release of the active ingredient in pH 6.8 phosphate buffer was 31% after 2 h; after 10 h, 79% of the active ingredient originally used had been released.

### Example 4

1600 g of copolymer and 400 g of piroxicam (melting point 199° C.) were weighed into a Turbula mixing vessel and mixed in a T10B Turbula mixer for 10 minutes. A reciprocating piston pump was used to feed Cremophor EL into the extruder.

The mixture was extruded under the following conditions:
Zone temperature of 1st barrel: 20° C.; 2nd barrel: 40° C.
Zone temperature from the 3rd barrel: 140° C.
Screw speed 100 rpm
Throughput: 800 g/h
Liquid feed rate: 50 g/h
Die diameter 3 mm

The solid solutions were examined by XRD and by DSC and were found to be amorphous. After 1 h in 0.1 normal HCl, 100% active ingredient had been released.

### Example 5

1500 g of copolymer, 100 g of Lutrol F68 and 400 g of itraconazole (melting point 166° C.) were weighed into a Turbula mixing vessel and mixed in a T10B Turbula mixer for 10 minutes.

The mixture was extruded under the following conditions:
Zone temperature of 1st barrel: 20° C.; 2nd barrel: 40° C.
Zone temperature from the 3rd barrel: 145° C.
Screw speed 100 rpm
Throughput: 800 g/h
Die diameter 3 mm

The solid solutions were examined by XRD and by DSC and were found to be amorphous. After 1 h in 0.1 normal HCl, 100% active ingredient had been released.

1. A dosage form comprising formulations of sparingly water-soluble active ingredients in amorphous form in a polymer matrix comprising polyether copolymers and at least one surfactant, wherein the polyether copolymers are obtained by free-radically initiated polymerization of a mixture of 30 to 80% by weight of N-vinyllactam, 10 to 50% by weight of vinyl acetate and 10 to 50% by weight of a polyether.

2. The dosage form of claim 1, wherein at least one surfactants has a hydrophilic-lipophilic balance value greater than 3.

3. The dosage form of claim 1, wherein at least one surfactant, comprises an anionic, cationic, amphiphilic, zwitterionic or nonionic surfactant.

4. The dosage form of claim 1, comprising at least one surfactant selected from the group consisting of alpha-tocopherol polyethylene glycol succinate, stearic acid, stearic salts, glyceryl monostearate, ethoxylated glyceryl monostearate, sorbitan laurate, sorbitan monooleate, Ceteareth-20, sodium laurylsulfate, docusate sodium, poloxamers, ethoxylated castor oil, hydrogenated ethoxylated castor oil, Macrogol fatty alcohol ethers, Macrogol fatty acid esters, Macrogol sorbitan fatty alcohol ethers and Macrogol sorbitan fatty acid esters.

5. The dosage form of claim 1, wherein the ratio of the polyether copolymer to the surfactant is between 60:40 and 99:1.

6. The dosage form of claim 1, wherein the ratio of the polyether copolymer to the surfactant is between 90:10 and 99.9:0.1.

7. A process for producing formulations for dosage forms of sparingly water-soluble active ingredients, the process comprising extruding a melt comprising a sparingly water-soluble active ingredient, a polyether copolymer and at least one surfactant, wherein the polyether copolymer is obtained by free-radically initiated polymerization of a mixture of 30 to 80% by weight of N-vinyllactam, 10 to 50% by weight of vinyl acetate and 10 to 50% by weight of a polyether into the polymer matrix in addition to the polyether copolymer.

8. The process of claim 7, wherein the melt is extruded at temperatures below the melting point of the active ingredient.

9. The process of claim 7, wherein the polyether copolymer and active ingredient are mixed in a twin-screw extruder.

10. The process of claim 7, wherein at least one surfactant has a hydrophilic-lipophilic balance value greater than 3.

11. The process of claim 7, wherein at least one surfactant is selected from the group consisting of alpha-tocopherol polyethylene glycol succinate, stearic acid, stearic salts, glyceryl monostearate, ethoxylated glyceryl monostearate, sorbitan laurate, sorbitan monooleate, Ceteareth-20, sodium laurylsulfate, docusate sodium, poloxamers, ethoxylated castor oil, hydrogenated ethoxylated castor oil, Macrogol fatty alcohol ethers, Macrogol fatty acid esters, Macrogol sorbitan fatty alcohol ethers and Macrogol sorbitan fatty acid esters.

12. The process of claim 7, wherein the ratio of the polyether copolymer to the surfactant is between 60:40 and 99:1.

13. The process of claim 7, wherein the ratio of the polyether copolymer to the surfactant is between 90:10 and 99.9:0.1

14. The dosage form of claim 1, wherein the polyether copolymers are obtained from:
i) 40 to 60% by weight of N-vinyllactam
ii) 15 to 35% by weight of vinyl acetate and
iii) 10 to 30% by weight of a polyether.

15. The dosage form of claim 1, wherein the polyether copolymers are obtained from:
i) 50 to 60% by weight of N-vinyllactam
ii) 25 to 35% by weight of vinyl acetate and
iii) 10 to 20% by weight of a polyether.

16. The dosage form of claim 1, wherein the N-vinyllactam comprises N-vinylcaprolactam, N-vinylpyrrolidone or mixtures thereof.

17. The dosage form of claim 1, wherein at least one surfactant has a hydrophilic-lipophilic balance value greater than 10.

18. The process of claim 7, wherein the polyether copolymers are obtained from:
i) 40 to 60% by weight of N-vinyllactam
ii) 15 to 35% by weight of vinyl acetate and
iii) 10 to 30% by weight of a polyether.

19. The process of claim 7, wherein the polyether copolymers are obtained from:
i) 50 to 60% by weight of N-vinyllactam
ii) 25 to 35% by weight of vinyl acetate and
iii) 10 to 20% by weight of a polyether.

20. The process of claim 7, wherein the N-vinyllactam comprises N-vinylcaprolactam, N-vinylpyrrolidone or mixtures thereof.
